(19) United States
(12) Patent Application Publication
(10) Pub. No.: US 2021/0265054 A1
(43) Pub. Date: Aug. 26, 2021

(54) WEARABLE HEALTH APPARATUS FOR THE COLLECTION OF WELLNESS DATA AND PROVIDING FEEDBACK THEREFROM TO THE WEARER

(71) Applicant: Circular, Orsay (FR)

(72) Inventors: Amaury Kosman, Orsay (FR); Adiasa Suharno, Rodez (FR); Laurent Bsalis, Paris (FR)

(21) Appl. No.: 16/797,038

(22) Filed: Feb. 21, 2020

(51) Int. Cl. G16H 40/60; G06F 1/16; G16H 20/30

(52) U.S. Cl. CPC G16H 40/60; G16H 20/30; G06F 1/163

(57) ABSTRACT

A health and wellness tracking ring that can be worn by a user at all times, that is unobtrusive and that collects health and wellness data about the user providing a means for the wearer to access and utilize the collected data for continuous monitoring and improvement of the wearer's health and wellness. The ring includes a control button that is easily accessible to the user while wearing. The ring includes replaceable outer shells and includes hardware and software that allow the user to communicate with software in a computer device such as a cell phone, table, personal computer or AI assistant.

### BACKGROUND OF THE INVENTION

### Field of the Invention

The field of this invention relates generally to the field of wearable health and fitness devices and more particularly toward wellness and smart rings, which is a ring that can be worn by a user at all times, that is unobtrusive and that collects health and wellness data about the user providing a means for the wearer to access and utilize the collected data for continuous monitoring and improvement of the wearer's health and wellness.

### Description of the Prior Art

Tracking of health and fitness information through the use of wearable data collection devices is known in the art in the form of devices that can wrap around a wearer's wrist or finger or other part of the body for the collection of data that the device can be programmed to receive, such as vital signs and the measurement of activities, such as steps walked or ran.

The problem with existing devices is primarily the fact that they are not worn at all times, leaving gaps in the data collection when the user is not wearing the device. As a result, the conclusions drawn from the data are poor and therefore not useful to the user. The user needs to be wearing the data collection device while asleep as well as while awake, at all sorts of occasions from casual to elegant. Prior art devices fall short of providing the means for the meaningful collection of data.

It is the object of the instant invention to provide a wearable data collection device that overcomes the shortcomings of the prior art.

### SUMMARY OF THE INVENTION

The device of the instant invention provides for a wearable ring that acts as a computing device that can collect data from the wearer when wearing the ring. The ring includes a main body part that is molded and made of plastic material on the outer surface. The inner surface of the ring, which contacts the skin on the finger of the wearer, is made of silicon material and includes a raised portion on the inner surface therebetween creating a circular cavity through the inner surface with a flat part above which the raised portion is situated. Under the raised protrusion on the flat part is contained a rigid-flex printed circuit board (PCB), an infrared and RED heart rate sensor, an accelerometer, a vibration motor, memory, an RF Transceiver IC which has near field communication capability, a curved rechargeable battery and contact pins for charging. The device includes external and interchangeable outer shells made of titanium and a coating.

The instant invention provides a wearable health tracker that is worn 24 hours day and is therefore collecting more and relevant data to make personal recommendations. The tracker is a ring that can be worn on the finger of the user and can therefore be collecting data at all times of the day or night.

The ring has an inner layer and an outer layer that is customizable and detachable. The inner layer contains the electronics that can communicate with smartphones and mobile devices, personal computers and AI assistants. The ring is programmable to collect data such as predictions of the user's energy level and recovery time, detection of long-term lifestyle trends of the user, provide day and night clinical data including sleep patterns, predict a weakening immune system and the onset of illness and silently wake the user with full energy in the morning.

The interchangeable outer shell can provide a variety of materials with which it is made, including durability, water resistance and aesthetic choices such as color. The inner ring includes the programmable electronics and the control in the form of buttons that extend upward through the outer ring.

The basic embodiment of the present invention teaches a wearable, wellness-tracking ring comprising: a main body portion that is circular in shape wherein said main body portion has an outer surface and inner surface wherein said outer surface of said main body portion contains a central groove created by a first flange and a second flange surrounding the perimeter of said main body portion on either side of said outer surface wherein an aperture is created therebetween that allows said main body portion to fit around the finger of a user; a removeable external outer shell to be affixed and secured to said outer surface inside of said groove; a raised portion along said inner surface of said main body that is covered by a protective top thereby creating a flat portion along said aperture created by said inner surface wherein between said protective top and said flat portion are housed electronic components to capture data from said user because said inner surface contacts the skin of said user; and a control button on said outer surface that is accessible to said user while said ring is being worn.

The above embodiment can be further modified by defining that said main body portion is made of plastic.

The above embodiment can be further modified by defining that said electronic components are taking from the following: rigid-flex printed circuit board, infrared and/or red heart rate sensor, curved rechargeable battery, contact pins for charging, accelerometer, vibration motor, flash memory, power management integrated circuit, Bluetooth microcontroller.

The above embodiment can be further modified by defining that an RF transceiver IC with near field communication capability is situated on said main body portion.

The above embodiment can be further modified by defining that said RF transceiver is situated proximate said control button.

The above embodiment can be further modified by defining that said data that is collected is communicated to a device, such as a cellphone, a tablet, a personal computer or an AI assistant.

The above embodiment can be further modified by defining that said flat portion, when worn, is situated near the bottom of said user's finger proximate said user's palm so that said control button is oriented toward the thumb of said user for easy access while worn.

The above embodiment can be further modified by defining that wherein software is installed in said cellphone, tablet, personal computer or AI assistant such that said software can utilize said data to provide feedback to said user.

The above embodiment can be further modified by defining that said feedback is taken from the following: making action recommendations for better health and wellness; provide alerts through said vibration motor such as alarms, reminders or alerting said user when said ring has traveled too far from said cell phone, tablet, personal computer or AI assistant.

The above embodiment can be further modified by defining that said control button can be utilized as an emergency signaler to said cell phone, table, personal computer or AI assistant.

The above embodiment can be further modified by defining that said ring and associated software is programmed to induce sleep, give indications, interact with the user and evoke relaxation through pace breathing and haptic feedback.

An alternate embodiment teaches a wearable, interactive, wellness-tracking ring with a vibration motor embedded therein.

### BRIEF DESCRIPTION OF THE DRAWINGS

For a better understanding of the present invention, reference is to be made to the accompanying drawings. It is to be understood that the present invention is not limited to the precise arrangement shown in the drawings.

FIG. 1 is an isometric view of the assembled ring of the instant invention.

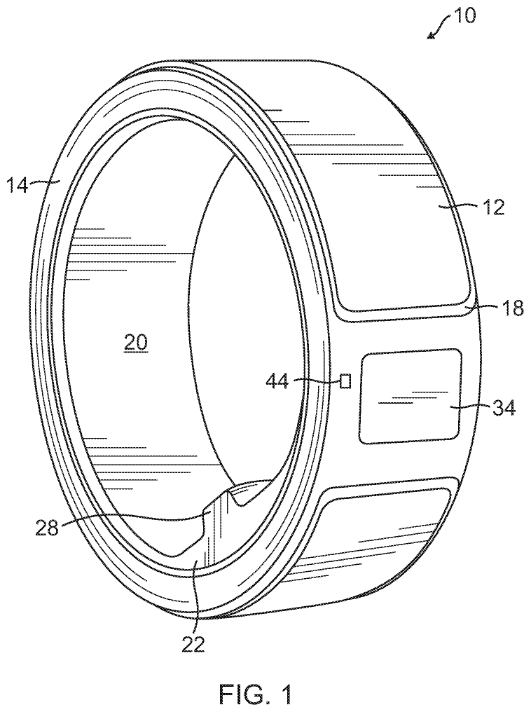

FIG. 2 is an exploded view of an illustrative embodiment of the ring of the instant invention showing the main body ring and two outer shells that attach thereto.

FIG. 3 is an isometric view of an illustrative outer shell for the ring of the instant invention.

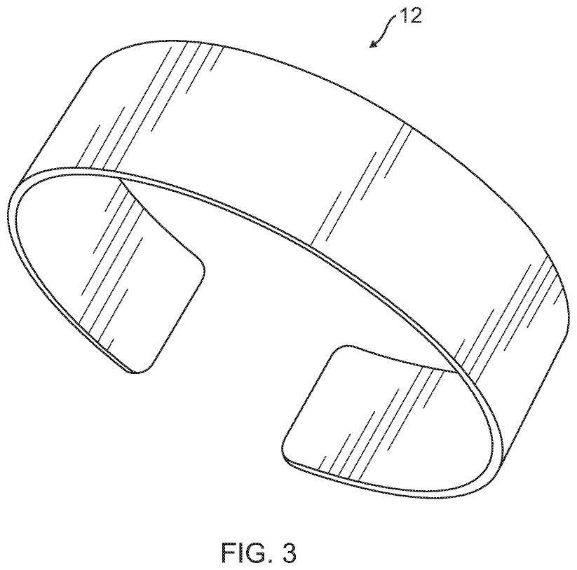

FIG. 4 is a close-up view of the heart rate sensor in the raised portion on the inner surface of the main body of the ring of the instant invention.

FIG. 5 is an illustrative view of the ring of the instant invention around the finger of a user showing how the user can access the power button on the outer surface of the main body of the ring.

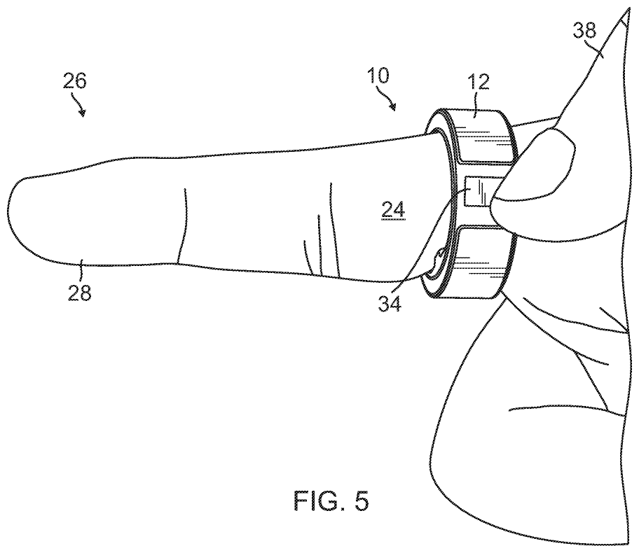

FIG. 6 is a view illustrating how the ring can charge on a power dock.

FIG. 7A is an isometric view of the ring of the instant invention showing the flat portion of the ring.

FIG. 7B is a side view of the ring of the instant invention showing the flat portion of the ring.

FIG. 8 is a schematic diagram outlining the electrical components contained in the flat portion of the ring of the instant invention.

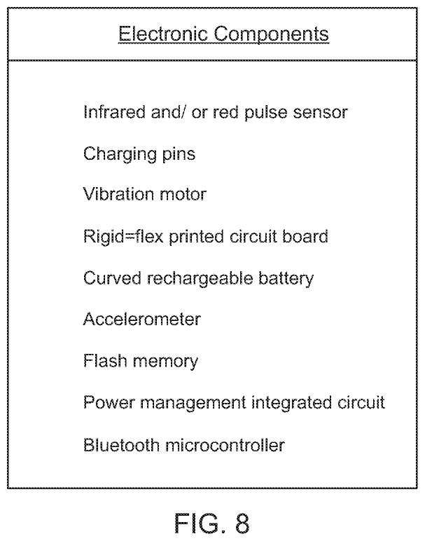

FIG. 9 is a table outlining some of the software functions included in communication with the ring of the instant invention.

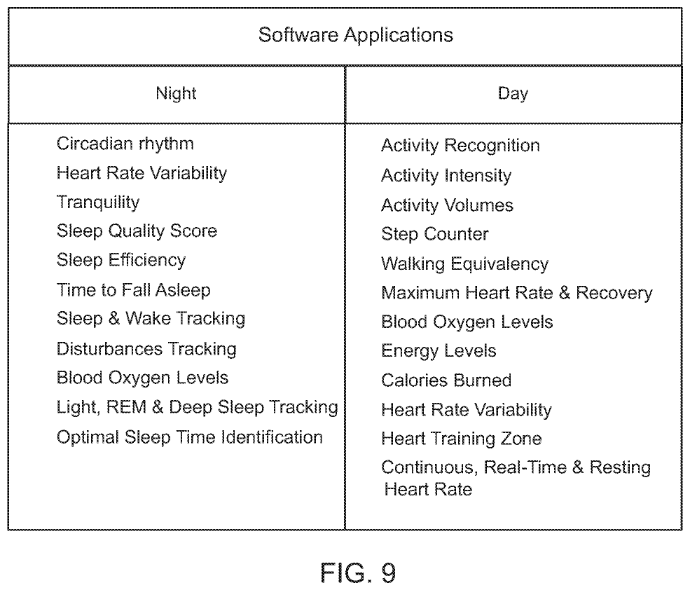

### DETAILED DESCRIPTION OF A PREFERRED EMBODIMENT

Turning to the drawings, the preferred embodiment is illustrated and described by reference characters that denote similar elements throughout the several views of the instant invention.

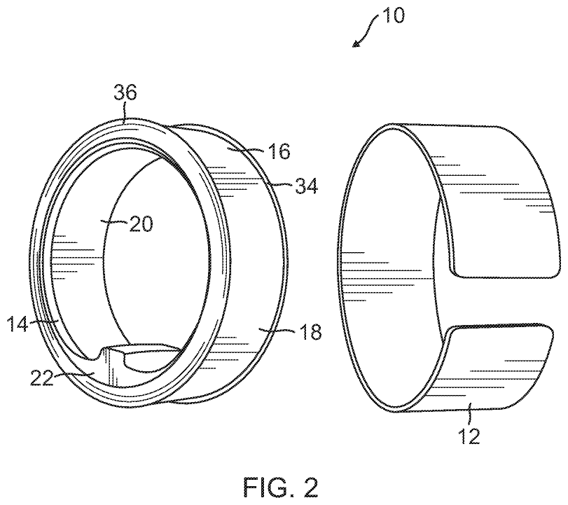

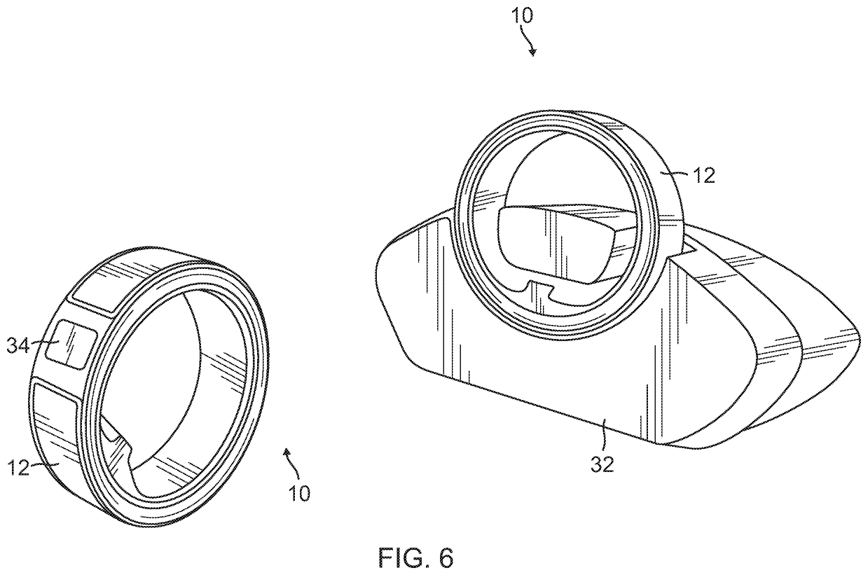

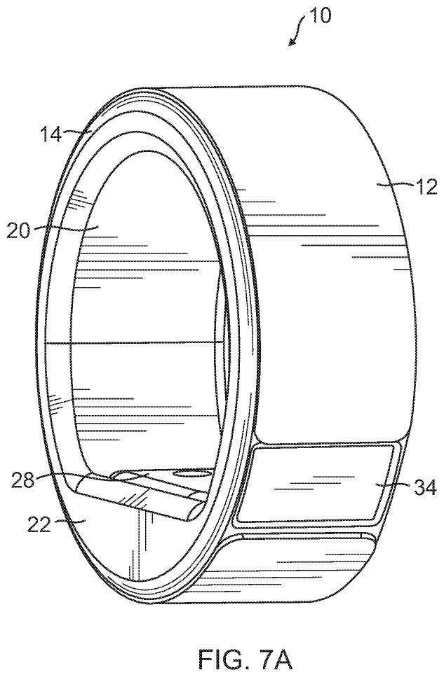

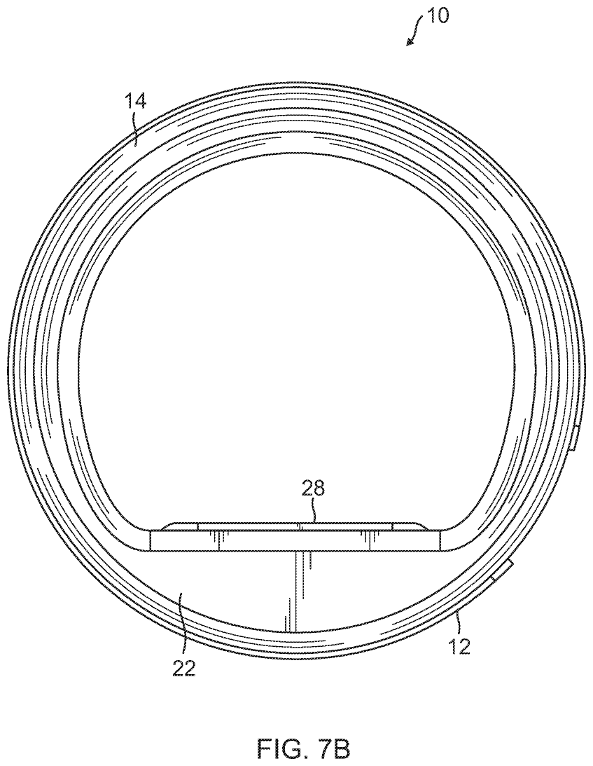

The ring 10 of the instant invention has two main parts. The first main part is the changeable outer shell 12, and the second part is the main body 14. The main body 14 breaks down into smaller parts. Viewing the ring 10 from the outside in (See FIG. 2), first is the outer shell 12, which is made out of plastic. The outer shell 12 serves as a protective outer portion to the assembled ring 10. The main body 14 includes a groove 16 on the outer surface 18 that provides a slot for insertion of the changeable outer shell 12. The main body 14 also has an inner surface 20 that contacts the skin 24 on the finger 28 of the wearer 26. The inner surface 20 is substantially circular around most of the inner circumference of the ring 10, but bottoms out in a flat portion 22 (See FIGS. 7A-7B). The flat portion 22 provides a platform into which the electronics and the assembled PCB are housed, covered with an overmold housing 28. This flat portion 22 platform also includes charging pins 30 that allow the ring 10 to be charged on a charging dock 32 (See FIG. 6.)

The ring 10 of the instant invention includes a button 34. This button 34 provides control the user and can be used to stop vibrations, to control other devices and to trigger any other actions programed into the electronics.

One feature of the instant invention is the customization achievable through the detachable outer shells 12. This allows the user to change the look as desired by the user. The outer shells 14 are preferably made of titanium and come in a variety of different colors. Decorative, aesthetic engravings and the inclusion of gems to accentuate the ring are contemplated by the design.

To change out the outer shell 12, the user simply slides it and/or pulls it to detach it from the rails 34, 36 of the outer shell 14 that define the groove 16. The user needs only to clip the outer shell 12 back in place.

The PCB rigid-flex structure is designed with components restricted such as RF antenna 44, button and, fitted inside of the resin mold or overmold housing 28 in order to have a solid and compact structure. RF antenna is placed outside the ring and near the button for optimal range of data transmission. The outer shell renders the ring totally waterproof even with a button and charging pins. The final assembly has a very specific direction and positioning design which is made for ease of use and better accuracy when worn, i.e., on the finger 28, the flat part 22 needs to be on the bottom of the finger 28 for better data accuracy and the button 34 needs to be turned towards the wearer's thumb 38 for easy clicking of the button 34 from any finger 28. This precise positioning (bottom of the finger, on the palm side) exploits the fact that the nervous system of the finger is much more sensitive than the one of the wrist which allows a better accuracy of the optical sensor and a better sensitivity to the vibrations of the ring compared to prior art devices that are wrapped around wrist.

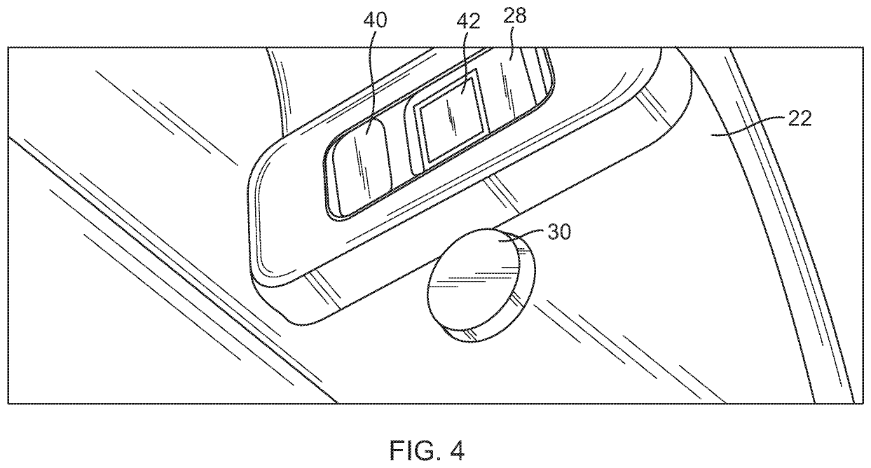

The flat portion 22 houses an infrared optical pulse sensor 40 (See FIG. 4), charging pins 30 and a vibration motor 40. The ring is 8 mm wide 2.8 mm thick and weighs only 4 grams making it one of the most discreet, seamless and lightweight wellness wearable technology devices.

The sensors allow for 24/7 tracking by recognizing specific gestures prior to activation, at all times, i.e., there is continuous wellness tracking for better recommendations by the software in the app housed in the mobile device 44. The software with the associated app allows for the calculation of various metrics, including, but not limited to: heart rate, heart rate variability, blood oxygenation levels (SpO2), steps taken, walking equivalency, kcal burned, active minutes, VO2 max, heart rate max, recovery levels, resting Heart rate, sleep balance, activity volume, activity intensity, sleep disturbances, real sleep, circadian rhythm, REM sleep, deep sleep, light sleep, sleep latency, sleep debt, hours slept. Furthermore, the software in the associated app can provide a calculation of unique scoring systems

Precise algorithms are established based on wearer's baseline, which is an innovative way of treating biometrics from sensors for a wearable device. Such algorithms allow the software to make recommendations to users in a unique and personal way through the establishment of the baselines for each metric mentioned above, for each user.

Other features of the device and associated app include an alarm clock that utilizes data transmitted from the ring to the user's phone in order to track above metrics and can provide a wake up in order to combat sleep inertia (“smart alarm”) (and/or manual alarm), or provide a wake ups that are in alignment with furthering wellness based on the data previously gathered. The ring automatically transmits data throughout the user's core sleep or any nap episode to calculate above metrics. The ring and associated app can provide reminders in the form of a ring vibration of a predefined event. The cross correlation of both day and night data gathering is unique to the product of the instant invention as it can be worn during both times.

The accuracy achieved by the 24/7 wearing of the instant invention is important in that it allows for more accurate and useful wellness recommendations. The performance result closely approximates laboratory level data treatment in existence for cardiac, sleep and activity science, which currently only exist in labs, which is prohibitively expensive for routine wellness as well as being very invasive.

The embedded vibration function can provide a medication reminder and can also provide a warning when the user gets too far from his phone. Additionally, when the ring is not connected, it vibrates. A curated list of activity and sleep programs take advantage of both the heart rate sensor and the embedded accelerometer so the user is reminded of every step to take during the program and whereas the sensors continuously transmit data to the user's phone.

Related to health, an arrhythmia detection algorithm can be programmed, taking full advantage of the heart rate sensor, whereas data is transmitted at very high frequencies to detect and prevent abnormal heart beat signatures. A tension measurement feature can be incorporated with similar principals. Likewise, a driving drowsiness detection features can alert a drowsy driver. A sleep apnea detection algorithm can take full advantage of the heart rate sensor and of the oxygenation levels datasets, whereas data is transmitted at specific frequencies to detect and prevent abnormal respiratory signatures.

The ring can include an emergency feature, whereas the user presses the ring's button (once or several times defined in the app), it transmits data the user's phone and cellular network to alert any other individual of an abnormal situation. This feature is customizable as a control feature wherein when the user presses the ring's button, it triggers a specific interaction determined beforehand, linking the ring through the user's phone to other smart objects and/any connected device. A gesture enabled feature can be incorporated wherein when a specific motion of the user's finger(s) and or hand occurs, an action is triggered.

The invention of the instant invention can, by design, also house technologies, including; wireless charging, RF energy harvesting technology, temperature measurement, near field communication, contactless payment, microphone integration, speaker integration.

The invention illustratively disclosed herein suitably may be practiced in the absence of any element which is not specifically disclosed herein.

The discussion included in this patent is intended to serve as a basic description. The reader should be aware that the specific discussion may not explicitly describe all embodiments possible and alternatives are implicit. Also, this discussion may not fully explain the generic nature of the invention and may not explicitly show how each feature or element can actually be representative or equivalent elements. Again, these are implicitly included in this disclosure. Where the invention is described in device-oriented terminology, each element of the device implicitly performs a function. It should also be understood that a variety of changes may be made without departing from the essence of the invention. Such changes are also implicitly included in the description. These changes still fall within the scope of this invention.

Further, each of the various elements of the invention and claims may also be achieved in a variety of manners. This disclosure should be understood to encompass each such variation, be it a variation of any apparatus embodiment, a method embodiment, or even merely a variation of any element of these. Particularly, it should be understood that as the disclosure relates to elements of the invention, the words for each element may be expressed by equivalent apparatus terms even if only the function or result is the same. Such equivalent, broader, or even more generic terms should be considered to be encompassed in the description of each element or action. Such terms can be substituted where desired to make explicit the implicitly broad coverage to which this invention is entitled. It should be understood that all actions may be expressed as a means for taking that action or as an element which causes that action. Similarly, each physical element disclosed should be understood to encompass a disclosure of the action which that physical element facilitates. Such changes and alternative terms are to be understood to be explicitly included in the description.

What is claimed is:

1. A wearable, wellness-tracking ring comprising:
a main body portion that is circular in shape wherein said main body portion has an outer surface and inner surface wherein said outer surface of said main body portion contains a central groove created by a first flange and a second flange surrounding the perimeter of said main body portion on either side of said outer surface wherein an aperture is created therebetween that allows said main body portion to fit around the finger of a user;
a removeable external outer shell to be affixed and secured to said outer surface inside of said groove;
a raised portion along said inner surface of said main body that is covered by a protective top thereby creating a flat portion along said aperture created by said inner surface wherein between said protective top and said flat portion are housed electronic components to capture data from said user because said inner surface contacts the skin of said user; and
a control button on said outer surface that is accessible to said user while said ring is being worn.

2. The ring as defined in claim 1 wherein said main body portion is made of plastic.

3. The ring as defined in claim 1 wherein said electronic components are taking from the following: rigid-flex printed circuit board, infrared and/or red heart rate sensor, curved rechargeable battery, contact pins for charging, accelerometer, vibration motor, flash memory, power management integrated circuit, Bluetooth microcontroller.

4. The ring as defined in claim 1 wherein an RF transceiver IC with near field communication capability is situated on said main body portion.

5. The ring as defined in claim 4 wherein said RF transceiver is situated proximate said control button.

6. The ring as defined in claim 3 wherein said data that is collected is communicated to a device, such as a cellphone, a tablet, a personal computer or an AI assistant.

7. The ring as defined in claim 1 wherein said flat portion, when worn, is situated near the bottom of said user's finger proximate said user's palm so that said control button is oriented toward the thumb of said user for easy access while worn.

8. The ring as defined in claim 6 wherein software is installed in said cellphone, tablet, personal computer or AI assistant such that said software can utilize said data to provide feedback to said user.

9. The ring as defined in claim 8 wherein said feedback is taken from the following: making action recommendations for better health and wellness; provide alerts through said vibration motor such as alarms, reminders or alerting said user when said ring has traveled too far from said cell phone, tablet, personal computer or AI assistant.

10. The ring as defined in claim 8 wherein said control button can be utilized as an emergency signaler to said cell phone, table, personal computer or AI assistant.

11. The ring as defined in claim 8 wherein said ring and associated software is programmed to induce sleep, give indications, interact with the user and evoke relaxation through pace breathing and haptic feedback.

12. A wearable, interactive, wellness-tracking ring with a vibration motor embedded therein.
